(19) United States
(12) Patent Application Publication
(10) Pub. No.: US 2005/0107320 A1
(43) Pub. Date: May 19, 2005

(54) Methods and compositions for use in interventional pharmacogenomics

(76) Inventor: Matthew During, New York, NY (US)

Correspondence Address: NUTTER MCCLENNEN & FISH LLP, WORLD TRADE CENTER WEST, 155 SEAPORT BOULEVARD, BOSTON, MA, 02210-2604, US

(21) Appl. No.: 10/769,182

(22) Filed: Jan. 30, 2004

Related U.S. Application Data
(60) Provisional application No. 60/443,648, filed on Jan. 30, 2003.

(51) Int. Cl. A61K 48/00

(52) U.S. Cl. 514/44; 424/93.2

(57) ABSTRACT

The present invention provides methods and compositions for the interventional pharmacogenomics. The invention is based modifying an environment in a subject that is non-receptive to a therapeutic agent, such that the increased expression of a heterologous protein that interacts with the therapeutic agent, produces an environment that is receptive to the therapeutic agent, thereby making the therapy efficacious in the subject.

### BACKGROUND OF THE INVENTION

The technical field of this invention is pharmacogenomics, and in particular, the application of pharmacogenomics to neurological or neurodegenerative diseases.

Several areas in drug development and transition to clinical trials have been disappointing in recent times. One group of agents that may be considered amongst the greatest underachievers are growth factors, in particular nerve growth factors or neurotrophic agents. Following discovery of the neurotrophins, including NGF, BDNF, NT-3, NT-4/5, and GDNF among others, there was expectation that these potent growth factors would soon be revolutionizing treatment of neurological disorders (Alberch et al. (2002) Brain Res Bull 57, 817-822; Pradat et al (2001) Hum Gene Ther 12, 2237-2249; Shinohara et al (2002) Proc Natl Acad Sci USA 99, 1657-1660; Smith et al (1999) Proc Natl Acad Sci USA 96, 10893-10898; and Zou et al (1999) Gene Ther 6, 994-1005. Several companies have pursued the use of these factors for neurodegenerative disorders including Alzheimer's, Parkinson's and ALS without success (Apfel (2001) Clin Chem Lab Med 39, 351-355 and Apfel (2002) Int Rev Neurobiol 50, 393-413). Recently, the approach of expressing neurotrophins directly in target tissues with viral vectors, and hence overcoming delivery problems, has become popular (Andsberg et al. (2002) Neurobiol Dis 9, 187-204 and Pradat et al (2001) Hum Gene Ther 12, 2237-2249).

These approaches rely on the assumption that the target tissue will be fully responsive to the growth factor. Hence, one of the limitations of using neurotrophic factors for the treatment of neurological disorders is that target cells need to express appreciable levels of receptors to be responsive. The lack of a responsive receptor subtype may result in the remarkably polarized effects observed in people upon growth factor treatment, with a subgroup responding extremely well, with other subgroups being only partially responsive or completely refractory. The current approach to this inconsistency of therapeutic efficacy has been to develop pharmacogenomic analyses, whereby the gene(s) and specific polymorphisms responsible for differential responsivity are identified and thereby the drug of interest is only given to those with the responder genotype (Maimone et al (2001) Eur J Pharmacol 413, 11-29). Accordingly, a need exists to provide an environment that is responsive to a therapeutic agent independent of the genotype.

### SUMMARY OF THE INVENTION

The present invention describes methods for modifying target tissues to make them responsive and thus render a subject, e.g., mammalian, e.g., human, a responder independent of genotype. The present invention is based on modifying an environment in a subject that is non-receptive to a therapeutic agent, such that the increased expression of a heterologous protein that interacts with the therapeutic agent in the environment, produces an environment that is receptive to the therapeutic agent, thereby making the therapy efficacious in the subject. In particular, the current invention involves somatic cell gene transfer of a specific gene to a target cell population in vivo thereby making such cells responsive to a locally or systemically delivered therapeutic agent. The gene transfer in itself has no significant therapeutic effects, but the transfer of the gene enables the transduced cells to respond to a given therapeutic agent such as a drug. Hence, this invention is termed “interventional pharmacogenomics,” whereby an individual who may be poorly responsive or completely unresponsive to a drug or biological agent is now rendered a responder by a gene transfer strategy. In particular, the invention is directed to the administration of a growth factor receptor to change the ability of a cell to respond to the appropriate growth factor independent of its genotype. The transduced cell will be phenotypically indistinguishable from untransduced cells in absence of the growth factor. Application of the growth factor will then induce a phenotypic response in the transduced target cells.

The invention uses a vector to stably transduce the target tissue or organ. The vector contains a transgene cassette for optimal expression of the appropriate growth factor receptor. Expression of the receptor will be under control of a constitutive promoter that is optimized for target tissue expression. Expression of the receptor will render a previously non-responsive population of cells responsive to the appropriate growth factor. When expression levels peak, the growth factor will be administered either systemically or locally. The cellular response can be regulated temporally or spatially by the targeted administration of the hormone.

Expression of high(er) levels of receptor may also decrease the concentration of ligand required to initiate a response compared to other known growth factor targets. For example, a tissue that was normally responsive to a high concentration of growth factor, at a level which had adverse affects on other organs/tissues at that concentration, may now be responsive to a much lower level of growth factor that may have less adverse side affects.

Accordingly, in one aspect the invention pertains to a method of inducing an efficacious phenotypic response to a therapeutic agent by introducing a vector comprising a gene into a mammalian cell, the gene being operably linked to a promoter functional in the mammalian cell and encodes a heterologous protein. The expression of the heterologous protein within the mammalian cell modifies the environment of the cell from an environment that is non-receptive to the therapeutic agent to an environment that is receptive to subsequent delivery of the therapeutic agent. A therapeutic agent is then delivered to the mammalian cell with the receptive environment, such that the therapeutic agent interacts with expressed heterologous protein and induces an efficacious phenotypic response to the therapeutic agent.

The heterologous protein can be a number of biological proteins such as receptors, enzymes, and carbohydrates. The therapeutic agent can be selected based on the type of biological protein that is being modified. For example, if the heterologous protein is a receptor, then the therapeutic agent can be a ligand for the receptor, or an agonist/antagonist for the receptor. Examples of therapeutic agents include, but are not limited to, ligand, an agonist, antagonist, and drug.

The heterologous protein can be delivered to the mammalian cell using a vector vehicle such as viral vectors, for example, adeno-associated viral vector, lentiviral vector, and adenoviral vector. In one embodiment, the vector is an adeno-associated viral vector selected from the serotype of AAV-1, AAV-2 AAV-3, AAV-4, AAAV-5, AAV-6, and AAV-7. In a preferred embodiment, the adeno-associated viral vector is AAV-2, or a modified form of AAV-2 with an altered tropism.

In another embodiment, the invention pertains to a method of inducing an efficacious phenotypic response to a therapeutic agent in a central nervous system of a subject by introducing a vector comprising a gene into a cell present in the central nervous system of the subject, the gene being operably linked to a promoter functional in the central nervous system and encodes a heterologous protein. The expression of the heterologous protein within the cell of the central nervous system modifies the environment of the central nervous system from an environment that is non-receptive to a therapeutic agent to an environment that is receptive to subsequent delivery of the therapeutic agent. The therapeutic agent is then delivered to the central nervous system, such that the therapeutic agent interacts with expressed heterologous protein and induces an efficacious phenotypic response to the therapeutic agent.

In yet another aspect, the invention pertains to a method of inducing an efficacious phenotypic response to a therapeutic ligand in a brain of a subject with a disorder, by introducing a vector comprising a gene into a region of the brain of a subject, the gene being operably linked to a promoter functional in the brain and encoding a heterologous receptor for the ligand. The expression of the receptor modifies the environment in the region of the brain from an environment that is non-receptive to the therapeutic ligand to an environment that is receptive to subsequent delivery of the therapeutic ligand. The therapeutic ligand is then delivered to the region of the brain, such that the therapeutic ligand interacts with expressed heterologous receptor and induces an efficacious phenotypic response to the therapeutic ligand.

In one embodiment, the heterologous receptor is a receptor is an erythropoietin receptor, and the ligand is erythropoietin. The method of the invention can be used to ameliorate a disorder such as a neurodegenerative or neurological disorder associated with the brain, particularly, Parkinson's disease.

In yet another aspect, the invention pertains to a method of personalizing medical intervention for a subject with a disorder, by determining the expression level of a receptor for a therapeutic ligand from a mammalian cell that requires medical intervention. The expression level is compared with the expression level of the receptor with a predetermined standard at which a therapeutic ligand is found to be efficacious. A vector comprising a gene encoding a protein that modifies the environment is introduced to the mammalian cell that requires medical intervention , the gene being operably linked to a promoter functional in the mammalian cell and encoding a heterologous receptor for the ligand, wherein the gene expresses the receptor for the ligand within the mammalian cell to a level of the predetermined standard, and renders modifies the environment in the mammalian cell from an environment that is non-receptive to the therapeutic ligand to an environment that is receptive to the therapeutic ligand. The therapeutic ligand is then delivered to the mammalian cell, such that the therapeutic ligand interacts with expressed heterologous receptor. The therapeutic effect of the ligand on the mammalian cell is measured, and the expression level of the receptor can be modified to provide personalized medical intervention for the subject. The therapeutic ligand can be a known ligand.

### DETAILED DESCRIPTION

The practice of the present invention employs, unless otherwise indicated, conventional methods of microbiology, molecular biology and recombinant DNA techniques within the skill of the art. Such techniques are explained fully in the literature. (See, e.g., Sambrook, et al. Molecular Cloning: A Laboratory Manual (Current Edition); DNA Cloning: A Practical Approach, Vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, Vol. I & II (P. Tijessen, ed.); Fundamental Virology, 2nd Edition, Vol. I & II (B. N. Fields and D. M. Knipe, eds.)). All publications, patents, and patent applications cited herein are hereby incorporated by reference.

The term “interventional pharmacogenomics” as used herein refers to rendering an subject who may be poorly responsive, or completely unresponsive to a therapeutic agent, drug or biological agent, responsive to the therapeutic agent by a gene transfer strategy.

The term “efficacious phenotype” or “efficacious phenotypic response” as used herein refers to the manifestation in a subject of the desired beneficial effect. For example, the manifestation of the desired beneficial effect upon administration of a therapeutic agent such as aspirin to relieve symptoms of pain. The efficacious phenotype can manifest in a subject who was previously non-responsive to a therapeutic agent, and did not exhibit an a desired therapeutic response to the therapeutic agent, but who has been made responsive by modifying the environment in the subject, e.g. in an organ or region of an organ, by delivering and expressing a heterologous protein that interacts with the therapeutic agent, such that expression of the heterologous protein renders the subject responsive to the therapeutic agent, thereby producing the desired efficacious phenotype.

The phrase “modifies the environment” as used herein refers to altering or changing an environment in a subject such that it becomes responsive to a therapeutic agent. the environment can be modified by expressing and increasing the numbers of heterologous proteins in the region. The phrase refers to the increase or decrease of a heterologous protein in an environment. Preferably, phrase refers to increase of the heterologous protein. The term modifies or modified also refers to up-regulation or down-regulation, the increase, decrease, elevation, or depression of processes or signal transduction cascade involving the heterologous protein. A heterologous protein, can be a receptor, for example, an erythropoietin receptor (EPO-R). Increased expression of the EPO-R in an environment that is low in the EPO-R, modifies the environment, and makes the environment receptive to erythropoietin, the therapeutic ligand of the EPO-R. In one embodiment, the modification of the environment can be a direct modification, for example by expressing the heterologous protein directly in the environment. In another embodiment, the modification of the environment can be a indirect modification, for example by introducing an compound that induces the up-regulation of a protein receptor. For example, an antibody or fragment thereof which activates an erythropoietin receptor. Activation of an EPO-R refers to one or more molecular processes which an EPO-R undergoes that result in transduction of a signal to the interior of a receptor-bearing cell, where the signal ultimately brings about one or more changes in cellular physiology. Cellular responses to an EPO-R activation are typically changes in the proliferation or differentiation of receptor-bearing cells. Receptor-bearing cells are typically erythroid progenitor cells. Once the environment has been modified, it is receptive to the application of a therapeutic agent, for example, systemic delivery of erythropoietin (EPO). Non-limiting examples of modifications includes modifications of morphological and functional processes, under- or over production or expression of a substance or substances.

The phrase “non-receptive to the therapeutic agent” as used herein refers to an environment that does not produce the desired response in a subject when a therapeutic amount of a therapeutic agent is delivered to the subject. An environment may be non-receptive because it expressed a low number of the native receptor that interacts with the therapeutic ligand, for example, a low number of EPO-R available to interact with the EPO. Alternatively, the environment may be non-receptive because it does not express the native receptor at all. In both situations, the environment can be made receptive to the therapeutic ligand by expressing the heterologous protein that interacts with the therapeutic ligand, such that the increased amount of the heterologous protein is available to interact with the therapeutic ligand.

There are a number of existing drug therapies that are therapeutic to a population of individuals, however, there is a subpopulation of individuals that remain to be non-receptive to the therapy and therefore not treated. These individuals can be mad responsive to the therapeutic agent by modifying the environment to increase the number of heterologous proteins available to interact with the therapeutic agent, which can subsequently be delivered. For example, a subpopulation of individuals do not respond to using growth factors to treat neurodegenerative disorders such as Alzheimer's disease, Parkinson's disease, and ALS (See Apfel (2001) Clin Chem Lab Med 39, 351-355 and Apfel (2002) Int Rev Neurobiol 50, 393-413).

The phrase “receptive” as used herein refers to an environment that is responsive, or is more conducive, to a therapeutic agent. The environment can be made to be receptive by modifying the environment such that it can interact with a therapeutic agent administered at a therapeutic dose. This can be accomplished by increasing the levels of heterologous protein that interacts with a therapeutic ligand in the environment.

The phrase “heterologous protein” as used herein refers to a polypeptide which is produced by recombinant DNA techniques, where the DNA encoding the polypeptide is inserted into a suitable expression vector which is delivered to a host cell where it expresses the heterologous protein. The increased expression of the heterologous protein changes the environment in, and surrounding the host cell, such that the is an increased number level of the heterologous protein available to interact with a therapeutic agent, thereby modifying the environment of a subject from one that is non-responsive to a therapeutic agent, to one that is responsive to the therapeutic agent. For example, Parkinson's disease is caused by degeneration of dopaminergic neurons projecting from the substantia nigra pars compacta (SNpc) to the striatum. Among those most vulnerable are the dopaminergic neurons of the substantia nigra pars compacta (SNpc, A8 and A9 cells). However, the dopaminergic cells immediately adjacent (medial) to the SNpc, the A10 cells of the VTA are relatively spared in Parkinson's disease. One possibility for this discrepancy relates to the differential expression of neurotrophin receptors with the medial nigral dopamine neurons (A10 cells) being richer in neurotrophin receptors than the lateral SNpc dopaminergic neurons (Nishio et al (1998) Neuror Prot 9, 2847-2851). The relatively low level of neurotrophin receptor expression would also make these cells resistant to therapy with a specific growth factor. Hence, the method of the invention can be used to transduce the vulnerable SNpc dopaminergic cell population with a receptor that is usually not expressed, or expressed in low abundance such that this target cell population that was not responsive, now becomes responsive due to the increase in receptor level at the target site. Following the increased expression of the receptor at the target site, protection in these cells can be induced following the systemic delivery of the relevant growth factor, which is able to interact with the increased number of expressed receptors at the target site. For example, a vector comprising the gene encoding an EPO-R can be delivered into non-responsive cells of the SNpc. Expression of the EPO-R in the cells of the SNpc modifies the environment and renders these cells responsive to EPO, the therapeutic ligand of the EPO-R. Thus, expression of EPO-R will promote cell survival of transduced neurons upon systemic application of EPO. The skilled artisan will appreciate that the scope of the invention includes any number of heterologous proteins that can be expressed at a target site to alter an environment from one that is non-responsive to a therapeutic agent, to one that is responsive the therapeutic agent.

In another embodiment, more than one heterologous protein can be delivered and expressed at the target site to render an environment that is non-responsive to more than one therapeutic agent, to one that is responsive to more than one therapeutic agent. For example, by expressing an EPO-R receptor and a dopamine receptor, such that the environment is made responsive to both erythropoietin dopamine. Accordingly, the invention pertains to the expression of at least two different heterologous proteins at the target site, at least three different heterologous proteins, least four different heterologous proteins, a least five different heterologous proteins, at least six different heterologous proteins, least seven different heterologous proteins, least eight different heterologous proteins, least nine different heterologous proteins, and at least ten different heterologous proteins at the target site. The invention also includes expressing one or more variants of the heterologous protein. For example, different types of EPO-R. Examples of a heterologous protein include, but are not limited to, a receptor, carbohydrate, enzyme, and the like.

The phrase “therapeutic agent” as used herein refers to compounds that produce a desired beneficial effect such as existing therapeutic drugs, new therapeutic compounds, drugs, anti-tumor agents, toxins and the like. The phrase “therapeutic agent” particularly refers to one member of a pair, where one member of the pair is the heterologous protein and the other member of the pair is a therapeutic agent for the heterologous protein. For example, the heterologous protein member can be a receptor e.g., EPO-R, and therapeutic agent member can be a ligand for the receptor, e.g., EPO.

The term “central nervous system” or “CNS” as used herein refers to the art recognized use of the term. The CNS pertains to the brain, cranial nerves and spinal cord. The CNS also comprises the cerebrospinal fluid, which fills the ventricles of the brain and the central canal of the spinal cord.

The term “subject” as used herein refers to any living organism in which an immune response is elicited. The term subject includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

The term “gene transfer” or “gene delivery” as used herein refers to methods or systems for inserting foreign DNA into host cells. Gene transfer can result in transient expression of non-integrated transferred DNA, extrachromosomal replication and expression of transferred replicons (e.g., episomes), or integration of transferred genetic material into the genomic DNA of host cells.

The term “transfection” is used herein refers to the uptake of foreign DNA by a cell. A cell has been “transfected” when exogenous DNA has been introduced inside the cell membrane. Such techniques can be used to introduce one or more exogenous DNA moieties, such as a nucleotide integration vector and other nucleic acid molecules, into suitable host cells. The term captures chemical, electrical, and viral-mediated transfection procedures.

The term “host cell” as used herein refers to, for example microorganisms, yeast cells, insect cells, and mammalian cells, that can be, or have been, used as recipients of an AAV helper construct, an AAV vector plasmid, an accessory function vector, or other transfer DNA. The term includes the progeny of the original cell which has been transfected. Thus, a “host cell” as used herein generally refers to a cell which has been transfected with an exogenous DNA sequence. It is understood that the progeny of a single parental cell may not necessarily be completely identical in morphology or in genomic or total DNA complement to the original parent, due to natural, accidental, or deliberate mutation.

The term “coding sequence” or a sequence which “encodes” a particular protein, as used herein refers to a nucleic acid sequence which is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

A “nucleic acid” sequence refers to a DNA or RNA sequence. The term captures sequences that include any of the known base analogues of DNA and RNA such as, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudo-uracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, 2-thiocytosine, and 2,6-diaminopurine.

The term “homology” or “identity” or “homologous” as used herein refers to the percentage of likeness between nucleic acid molecules. To determine the homology or percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identify” is equivalent to amino acid or nucleic acid “homology”). The percent identify between the two sequences is a function of the number of identical positions shared by the sequences, taking into account the number of gaps, and the length of each gap, which need to be introduced for optimal alignment of the two sequences.

The comparison of sequences and determination of percent identify between two sequences can be accomplished using a mathematical algorithm. For example, the percent identity between two amino acid sequences can be determined using the Needleman and Wunsch ((1970) J. Mol. Biol. (48):444-453) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, and 6. In another example, the percent identify between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In yet another example, the percent identify between two amino acid or nucleotide sequences is determined using the algorithm of E. Meyers and W. Miller (CABIOS, 4:11-17(1989)) which has been incorporated into the ALIGN program (version 2.0), using a PAM120 weight residue table, a gap length penalty of 12 and a gap penalty.

I. Interventional Pharmacogenomics

In one aspect, the invention pertains to methods for modifying an environment in a subject from one that is non-responsive to a therapeutic agent, to one that is responsive to the therapeutic agent. An environment can be made responsive by introducing a heterologous protein into a target region in a subject that requires modifying. This can be accomplished by using a delivery vectors carrying a gene encoding the heterologous protein, expressing the heterologous gene at the target, and then subsequently delivering a therapeutic agent to the target region. The process is referred to as “interventional pharmacogenomics”, whereby an subject who may be poorly responsive, or completely unresponsive to a therapeutic agent, drug or biological agent, is now rendered responsive to the therapeutic agent.

II Heterologous Proteins

In one aspect, the invention pertains to modifying an environment in a subject from one that is non-receptive to a therapeutic agent, to one that is receptive to the therapeutic agent by expressing at least one heterologous protein at the target site. In a preferred embodiment, the environment is in the central nervous system of a subject, for example, a region of the spinal cord or a region of the brain. In another embodiment, the environment is in an organ of a subject. Examples of organs, include, but are not limited to, heart, kidney, liver, pancreas, and the like.

The environment can be modified by increasing the level of the heterologous protein in the environment, for example by delivering a vector carrying the nuclei acid encoding a heterologous protein to the target region. The heterologous protein can be expressed in all, or part of the organ. The heterologous protein can be expressed in the target region to modify the environment such that it is receptive to a therapeutic agent. In one embodiment, the heterologous protein is a neurotrophic factor. Neurotrophic factors play a physiological role in the development and regulation of neurons in mammals. In adults, basal forebrain cholinergic neurons, motor neurons and sensory neurons of the CNS retain responsiveness to neurotrophic factors and can regenerate after loss or damage in their presence. For this reason, neurotrophins are suitable as drugs for the treatment of neurodegenerative conditions such as Alzheimer's Disease (AD), Parkinson's Disease (PD), amyotrophic lateral sclerosis (ALS), peripheral sensory neuropathies and spinal cord injuries.

In another embodiment, the heterologous protein is a growth factor such as erythropoietin (See Section III). Other examples of heterologous proteins include, but are not limited to enzymes, carbohydrates, neurotransmitters and the like. The modification of the environment to render it responsive to a therapeutic agent can result in amelioration of a number of neurodegenerative conditions such as:

(i) Parkinson's Disease

In one embodiment, Parkinson's disease can be ameliorated using the method of the invention. Parkinson's disease (PD) is characterized behaviorally by motor disturbances such as tremor, rigidity and akinesia. Parkinson's disease is caused by degeneration of dopaminergic neurons projecting from the substantia nigra pars compacta (SNpc) to the striatum. Among those most vulnerable are the dopaminergic neurons of the substantia nigra pars compacta (SNpc, A8 and A9 cells). Of interest, the dopaminergic cells immediately adjacent (medial) to the SNpc, the A10 cells of the VTA are relatively spared in Parkinson's disease. One possibility for this discrepancy relates to the differential expression of neurotrophin receptors with the medial nigral dopamine neurons (A10 cells) being richer in neurotrophin receptors than the lateral SNpc dopaminergic neurons (Nishio et al (1998) Neurorprot 9, 2847-2851). The relatively low level of neurotrophin receptor expression would also make these cells resistant to therapy with a specific growth factor. Hence, this example of the invention is to transduce the vulnerable SNpc dopaminergic cell population with a receptor that is usually not expressed, or expressed in low abundance such that the target cell population is not fully responsive, and then to induce protection in these cells following the systemic delivery of the relevant growth factor. PD can be tested using the models described in the examples section. Ameliorative effects can be determined by immunohistochemistry analysis and changes in behavioural analysis (See examples section).

(ii) Alzheimer's Disease

In another embodiment, Alzheimer's disease can be ameliorated using the method of the invention, using known models of Alzheimer's disease. In subjects with neurodegenerative diseases such as Alzheimer's Disease (AD), neurons in the Ch4 region (nucleus basalis of Meynert) which have nerve growth factor (NGF) receptors undergo marked atrophy as compared to normal controls (see, e. g., Kobayashi, et al., Mol. Chem. Neuropathol., 15:193-206 (1991); Higgins and Mufson, Exp. Neurol., 106:222-236 (1989); Mufson, et al, Exp. Neurol, 105:221-232 (1989) and, Mufson and Kordower, Prog. Clin. Biol. Res., 317:401-414 (1989)). In normal subjects, NGF prevents sympathetic and sensory neuronal death during development and prevents cholinergic neuronal degeneration in adult rats and primates (Tuszynski, et al., Gene Therapy, 3:305-314 (1996)). The resulting loss of functioning neurons in this region of the basal forebrain is believed to be causatively linked to the cognitive decline experienced by subjects suffering from neurodegenerative conditions such as AD (Tuszynski, et al., supra and, Lehericy, et al., J Comp. Neurol., 330:15-31 (1993)). AD can be tested using the models described in the examples section. Ameliorative effects can be determined by immunohistochemistry analysis and changes in behavioural analysis (See examples section).

(iii) Amyloid Lateral Sclerosis (ALS)

Several models of amyloid lateral sclerosis are available. Mutations in the superoxide dismutase gene 1 (SOD-1) are found in patients with familial amyotrophic lateral sclerosis (FALS). Overexpression of a mutated human SOD-1 gene in mice results in neurodegenerative disease as result of motor neuron loss in lumbar spinal cord, providing a suitable model for FALS (See e.g., Mohajeri et al. (1998) Exp Neurol 150:329-336). Transgenic models of ALS are also described (See e.g., Gurney (1997) J Neurol Sci 152:S67-73). Expression of mutant SOD1 genes in transgenic mice causes a progressive paralytic disease whose general features resemble ALS in humans. These models can be used in the methods of the invention. A gain-of-function in these models can monitored, for example, improvement in motor impairments of the animal's limbs.

(iv) Huntington's disease

In another embodiment, Huntington's disease can be ameliorated using the method of the invention. Models of neurodegenerative diseases in several different animals have been developed. For example, rat (Isacson et al. (1985) Neuroscience 16:799-817), monkey (Kanazawa, et al. (1986) Neurosci. Lett. 71:241-246), and baboon (Hantraye. et al. (1992) Proc. Natl. Acad. Sci. USA 89:4187-4191; Hantraye,. et al. (1990) Exp. Neurol. 108:91-014; Isacson, et al.(1989) Exp. Brain Res. 75(1):213-220) models of Huntington's disease have been described in which effective therapies are predictive of therapeutic efficacy in humans. Neurodegeneration in Huntington's disease typically involves degeneration in one or both nuclei forming the stratium or corpus stratium, the caudate nucleus and putamen.

Modifying a region of the brain to make it more receptive to a therapeutic agent, and then subsequently delivering a therapeutic agent to the region may ameliorate Huntington's disease. To assess therapeutic strategies, the methods of the invention can beemployed as described in the examples section using an animal model, and inducing in a state resembling Huntington's diseases. Morphological and immunohistochemical studies can then be performed by conventional techniques to determine whether the method of the invention provides protection by assessing, both morphologically and functionally of the tissue. Behavioral tests can also be performed using standard techniques described in the examples.

For therapy of neurodegenerative disease in humans, an appropriate region of the basal forebrain can be treated with a neurotrophic factor. Within the targeted region, a neurotrophic factor is preferably delivered into 5 to 10 separate sites, depending on the condition treated. For example, in human AD, basal forebrain neuronal loss occurs over an intraparenchymal area of approximately 1 cm in diameter. To treat affected neurons over such a large region, the vector carrying the nucleic acid encoding the neurotrophic factor can be delivered to multiple sites, e.g., 10 separate sites. However, in treating localized injuries to the basal forebrain, the affected areas of the brain will likely be smaller such that selection of fewer sites (e. g., 5 or fewer) will be sufficient for restoration of a clinically significant number of cholinergic neurons.

III Erythropoietin (EPO) and the Ervthropoietin Receptor (EPO-R)

In a preferred embodiment the invention pertains to ameliorating Parkinson's disease by expressing EPO-R, and subsequently administering EPO to the subject. Erythropoietin (EPO) is a glycoprotein hormone involved in the growth and maturation of erythroid progenitor cells into erythrocytes. EPO is produced by the liver during fetal life and by the kidney of adults and stimulates the production of red blood cells from erythroid precursors. Decreased production of EPO, which commonly occurs in adults as a result of renal failure, leads to anemia. EPO has been produced by genetic engineering techniques involving expression and secretion of the protein from a host cell transfected with the gene encoding erythropoietin. Administration of recombinant EPO has been effective in the treatment of anemia. For example, Eschbach et al. (N. Engl J Med 316, 73 (1987)) describe the use of EPO to correct anemia resulting from chronic renal failure.

While it is clear that EPO activates cells to grow and/or differentiate by binding to specific cell surface receptors, the specific mechanism of activation as well as the structure of the receptor and any associated protein(s) is not completely understood. The erythropoietin receptor (EPO-R) is thought to exist as a multimeric complex. Sedimentation studies suggested its molecular weight is 330+48 kDa (Mayeux et al. Eur. J. Biochem. 194, 271 (1990)). Crosslinking studies indicated that the receptor complex consists of at least two distinct polypeptides, a 66-72 kDa species, and 85 and 100 kDa species (Mayeux et al. J. Biol. Chem. 266, 23380 (1991)); McCaffery et al. J. Biol. Chem. 264, 10507 (1991)). A distinct 95 kDa protein was also detected by immunoprecipitation of EPO receptor (Miura & Ihle Blood 81, 1739 (1993)). Another crosslinking study revealed three EPO containing complexes of 110, 130 and 145 kDa. The 110 and 145 kDa complexes contained EPO receptor since they could be immunoprecipitated with antibodies raised against the receptor (Miura & Ihle, supra).

Further insight into the structure and function of the EPO receptor complex was obtained upon cloning and expression of the mouse and human EPO receptors (D'Andrea et al. Cell 57, 277 (1989); Jones et al. Blood 76, 31 (1990); Winkelmann et al. Blood 76, 24 (1990); PCT Application No. WO90/08822; U.S. Pat. No. 5,278,065 to D'Andrea et al.) The full-length human EPO receptor is a 483 amino acid transmembrane protein with an approximately 224 amino acid extracellular domain and a 25 amino acid signal peptide. The human receptor shows about an 82% amino acid sequence homology with the mouse receptor. The cloned full length EPO receptor expressed in mammalian cells (66-72 KDa) has been shown to bind EPO with an affinity (100-300 nM) similar to that of the native receptor on erythroid progenitor cells. Thus this form is thought to contain the main EPO binding determinant and is referred to as the EPO receptor. The 85 and 100 KDa proteins observed as part of a cross-linked complex are distinct from the EPO receptor but must be in close proximity to EPO because EPO can be crosslinked to them. The 85 and 100 KDa proteins are related to each other and the 85 KDa protein may be a proteolytic cleavage product of the 100 KDa species (Sawyer J. Biol. Chem. 264, 13343 (1989)).

A soluble (truncated) form of the EPO receptor containing only the extracellular domain has been produced and found to bind EPO with an affinity of about 1 nM, or about 3 to 10-fold lower than the full-length receptor (Harris et al. J. Biol. Chem. 267, 15205 (1992); Yang & Jones Blood 82, 1713 (1993)). The reason for the reduced affinity as compared to the full length protein is not known. There is a possibility that other protein species may also be part of the EPO-R complex and contribute to EPO binding thus increasing the affinity. In support of this possibility is the observation of Dong & Goldwasser (Exp. Hematol. 21, 483 (1993)) that fusion of a cell line with a low affinity EPO receptor with a CHO cell which does not bind EPO resulted in a hybrid cell line exhibiting high EPO binding affinity of the receptor for EPO. In addition, transfection of a full length EPO-R into CHO cells resulted in a cell line with both high and low affinity receptors as measured by Scatchard analysis. Amplification of the EPO-R copy number increased the low affinity but not high affinity binding. These results are consistent with the presence of a limited quantity of a protein present in CHO cells that converts the low affinity EPO-R to high affinity.

Activation of the EPO receptor results in several biological effects. Three of the activities include stimulation of proliferation, stimulation of differentiation and inhibition of apoptosis (Liboi et al. Proc. Natl. Acad. Sci. USA 90, 11351 (1993); Koury Science 248, 378 (1990)). The signal transduction pathways resulting in stimulation of proliferation and stimulation of differentiation appear to be separable (Noguchi et al. Mol. Cell. Biol. 8, 2604 (1988); Patel et al. J. Biol. Chem. 267, 21300 (1992); Liboi et al. ibid). Some results suggest that an accessory protein may be necessary for mediating the differentiation signal (Chiba et al. Nature 362, 646 (1993); Chiba et al. Proc. Natl. Acad. Sci. USA 90, 11593 (1993)). However there is controversy regarding the role of accessory proteins in differentiation since a constitutively activated form of the receptor can stimulate both proliferation and differentiation (Pharr et al. Proc. Natl. Acad. Sci. USA 90, 938 (1993)).

IV Therapeutic Agents

In one aspect, the invention pertains to ameliorating a condition in a subject by modifying an environment in a subject from one that is non-receptive to a therapeutic agent, to one that is receptive to the therapeutic agent, and then subsequently delivering the therapeutic agent to the subject.

In one embodiment, the therapeutic agent is erythropoietin. EPO is a glycoprotein hormone involved in the growth and maturation of erythroid progenitor cells into erythrocytes. EPO is produced by the liver during fetal life and by the kidney of adults and stimulates the production of red blood cells from erythroid precursors. Decreased production of EPO, which commonly occurs in adults as a result of renal failure, leads to anemia. EPO has been produced by genetic engineering techniques involving expression and secretion of the protein from a host cell transfected with the gene encoding erythropoietin. Administration of recombinant EPO has been effective in the treatment of anemia. For example, Eschbach et al. (N. Engl J Med 316, 73 (1987)) describe the use of EPO to correct anemia resulting from chronic renal failure.

The purification of human urinary EPO was described by Miyake et al. (J. Biol. Chem. 252, 5558 (1977)). The identification, cloning, and expression of genes encoding erythropoietin is described in U.S. Pat. No. 4,703,008 to Lin. A description of a method for purification of recombinant EPO from cell medium is included in U.S. Pat. No. 4,667,016 to Lai et al.

Examples of other therapeutic agents include, but are not limited to, nerve growth factors (NGFs) such as a primary nerve growth factor (NGF), brain-derived neurotrophic factor (BDNF), neurotrophin 3 (NT3), neurotrophin 4/5 (NT-4/5), neurotrophin 6 (NT-6), ciliary neurotrophic factor (CNTF), glial cell line-derived neurotrophic factor (GDNF), fibroblast growth factor 2 (FGF-2), leukemia inhibitory factor (LIF) and certain members of the insulin-like growth factor family (e. g., IGF-1). NGF and NT3 in particular have been tested with promising results in clinical trials and animal studies (see, e. g., Hefti and Weiner, Ann Neurol., 20:275-281 (1986); Tuszynki and Gage, Ann. Neurol., 30:625-636 (1991); Tuszynski, et al., Gene Therapy, 3:305-314 (1996) and Blesch and Tuszynski, Clin. Neurosci., 3:268-274 (1996)). Of the known nerve growth factors, β-NGF (for treatment of the Ch4, as in AD) and GNGF (for treatment of the substantia nigra, as in PD) can be used in the method of the invention. In a preferred embodiment, the growth factor is EPO.

V Vectors for Delivery the Heterologous Protein

The vectors can be any vector suitable for delivering the nucleic acid encoding a heterologous protein to a host cell at the target site. The term “vector” as used herein refers to any genetic element, such as a plasmid, phage, Itransposon, cosmid, chromosome, artificial chromosome, virus, virion, etc., which is capable of replication when associated with the proper control elements and which can transfer gene sequences between cells. Thus, the term includes cloning and expression vehicles, as well as viral vectors. In a preferred embodiment, the invention uses adeno-associated viral vectors. AAV vectors can be constructed using known techniques to provide at least the operatively linked components of control elements including a transcriptional initiation region, a exogenous nucleic acid molecule, a transcriptional termination region and at least one post-transcriptional regulatory sequence. The control elements are selected to be functional in the targeted cell. The resulting construct which contains the operatively linked components is flanked at the 5′ and 3′ region with functional AAV ITR sequences.

The nucleotide sequences of AAV ITR regions are known. The AAV ITRs are regions found at each end of the AAV genome which function together in cis as origins of DNA replication and as packaging signals for the viral genome. AAV ITRs, together with the AAV rep coding region, provide for the efficient excision and rescue from, and integration of a nucleotide sequence interposed between two flanking ITRs into a mammalian cell genome. The ITR sequences for AAV-2 are described, for example by Kotin et al. (1994) Human Gene Therapy 5:793-801; Berns “Parvoviridae and their Replication” in Fundamental Virology, 2nd Edition, (B. N. Fields and D. M. Knipe, eds.) The AAV-2 ITR have 145 nucleotides the terminal 125 nucleotides of each ITR form palindromic hairpin (HP) structures that serve as primers for AAV DNA replication. Each ITR also contains a stretch of 20 nucleotides, designated the D sequence, which is not involved in hairpin structure formation. (See e.g., Wang et al. (1998) J. Virol. 72: 5472-5480 and Wang et al. (1997) J. Virol. 71: 3077-3082). Regions of the inverted terminal repeats (ITR) are designated as A, B, C, A′ and D at the 5′-end of the sequences and as D, A′, B/C, C/B and A at the 3′-end of the sequences. The site between these regions is referred to as the terminal resolution site, which serves as a cleavage site in the ITRs. For example, the Rep 78 and Rep 68 possess a number of biochemical activities which include binding the viral inverted terminal repeats (ITRs), nicking at the terminal resolution site, and helicase activity. (See e.g., Kotin (1994) Hum. Gene Therap. 5:793-801 and Muzycza et al. (1992) 158: 97-129).

The skilled artisan will appreciate that AAV ITR's can be modified using standard molecular biology techniques. Accordingly, AAV ITR's used in the vectors of the invention need not have a wild-type nucleotide sequence, and may be altered, e.g., by the insertion, deletion or substitution of nucleotides. Additionally, AAV ITR's may be derived from any of several AAV serotypes, including but not limited to, AAV-1, AAV-2, AAV-3, AAV-4, AAV-5, AAV-6, AAVX7, and the like. Furthermore, 5′ and 3′ ITR's which flank a selected nucleotide sequence in an AAV expression vector need not necessarily be identical or derived from the same AAV serotype or isolate, so long as the ITR's function as intended, i.e., to allow for excision and replication of the bounded nucleotide sequence of interest when AAV rep gene products are present in the cell.

The AAV rep coding region refers to a region of the AAV genome which encodes the replication proteins of the virus which are required to replicate the viral genome and to insert the viral genome into a host genome during latent infection (Muzyczka, (1992) Current Topics in Microbiol. and Immunol.; Bems, “Parvoviridae and their Replication” in Fundamental Virology, 2d ed., (B. N. Fields and D. M. Knipe, eds.). The term also includes functional homologues thereof such as the human herpesvirus 6 (HHV-6) rep gene which is also known to mediate AAV-2 DNA replication. The rep coding region, as used herein, can be derived from any viral serotype. The region need not include all of the wild-type genes but may be altered, e.g., by the insertion, deletion or substitution of nucleotides, so long as the rep genes function as intended.

The AAV cap coding region refers to a region of the AAV genome which encodes the coat proteins of the virus which are required for packaging the viral genome. The AAV cap coding region, as used herein, can be derived from any AAV serotype. The region need not include all of the wild-type cap genes but may be altered, e.g., by the insertion, deletion or substitution of nucleotides, so long as the genes provide for sufficient packaging functions when present in a host cell along with an AAV vector.

The AAV vectors are derived from an adeno-associated virus serotype, including without limitation, AAV-1, AAV-2, AAV-3, AAV-4, AAV-5, AAV6, etc. AAV vectors can have one or more of the AAV wild-type genes deleted in whole or part, preferably the rep and/or cap genes, but retain functional flanking ITR sequences. Functional ITR sequences are necessary for the rescue, replication and packaging of the AAV virion. Thus, an AAV vector is defined herein to include at least those sequences required in cis for replication and packaging (e.g., functional ITRs) of the virus. The ITRs need not be the wild-type nucleotide sequences, and may be altered, e.g., by the insertion, deletion or substitution of nucleotides, so long as the sequences provide for functional rescue, replication and packaging.

The vectors can be produced using “AAV helper functions” or “helpers” which refer to AAV-derived coding sequences that can be expressed to provide AAV gene products that, in turn, function in trans for productive AAV replication. Thus, AAV helper functions include the rep and cap regions. The rep expression products have been shown to possess many functions, including, among others: recognition, binding and nicking of the AAV origin of DNA replication; DNA helicase activity; and modulation of transcription from AAV (or other heterologous) promoters. The cap expression products supply necessary packaging functions. AAV helper functions are used herein to complement AAV functions in trans that are missing from AAV vectors.

An AAV helper construct refers generally to a nucleic acid molecule that includes nucleotide sequences which provide AAV functions. These AAV functions include the rep and cap coding regions that are replaced by a nucleotide sequence of interest in an AAV delivery vector. AAV helper constructs are commonly used to provide transient expression of AAV rep and/or cap genes to complement missing AAV functions that are necessary for lytic AAV replication; however, all previous helper constructs lack AAV ITRs and can neither replicate nor package themselves. AAV helper constructs can be in the form of a plasmid, phage, transposon, cosmid, virus, or virion. A number of other vectors have been described which encode Rep and/or Cap expression products. See, e.g., U.S. Pat. No. 5,139,941. The helper constructs of present invention include at least one copy of AAV ITR or functional equivalent to make it competent for AAV replication and rescue.

It may also be necessary to provide “accessory functions” which refer to non-AAV derived viral and/or cellular functions upon which AAV is dependent for its replication (Carter, (1990) “Adeno-Associated Virus Helper Functions,” in CRC Handbook of Parvoviruses, vol. I (P. Tijssen, ed.)). Thus, the term captures DNAs, RNAs and protein that are required for AAV replication, including those moieties involved in activation of AAV gene transcription, stage specific AAV mRNA splicing, AAV DNA replication, synthesis of Cap expression products and AAV capsid assembly. Viral-based accessory functions can be derived from any of the known helper viruses such as adenovirus, herpesvirus (other than herpes simplex virus type-1) and vaccinia virus.

Accessory functions can be provided by “accessory function vector” which refer generally to a nucleic acid molecule that includes nucleotide sequences providing accessory functions. An accessory function vector can be transfected into a suitable host cell, wherein the vector is then capable of supporting AAV virion production in the host cell. Expressly excluded from the term are infectious viral particles as they exist in nature, such as adenovirus, herpesvirus or vaccinia virus particles. Thus, accessory function vectors can be in the form of a plasmid, phage, transposon, cosmid or virus that has been modified from its naturally occurring form.

The skilled artisan can appreciate that regulatory sequences can often be provided from commonly used promoters derived from viruses such as, polyoma, Adenovirus 2, cytomegalovirus and Simian Virus 40. Use of viral regulatory elements to direct expression of the protein can allow for high level constitutive expression of the protein in a variety of host cells. Ubiquitously expressing promoters can also be used include, for example, the early cytomegalovirus promoter Boshart et al. (1985) Cell 41:521-530, herpesvirus thymidine kinase (HSV-TK) promoter (McKnight et al. (1984) Cell 37: 253-262), β-actin promoters (e.g., the human β-actin promoter as described by Ng et al. (1985) Mol. Cell Biol. 5: 2720-2732) and colony stimulating factor-1 (CSF-1) promoter (Ladner et al. (1987) EMBO J. 6: 2693-2698).

Alternatively, the regulatory sequences of the AAV vector can direct expression of the transgene preferentially in a particular cell type, i.e., tissue-specific regulatory elements can be used. Non-limiting examples of tissue-specific promoters which can be used include, central nervous system (CNS) specific promoters such as, neuron-specific promoters (e.g., the neurofilament promoter; Byrne and Ruddle (1989) Proc. Natl. Acad. Sci. USA 86:5473-5477) and glial specific promoters (Morii et al. (1991) Biochem. Biophys Res. Commun. 175: 185-191).

The AAV vector harboring the transgene flanked by AAV ITRs, can be constructed by directly inserting the transgene into an AAV genome which has had the major AAV open reading frames (“ORFs”) excised therefrom. Other portions of the AAV genome can also be deleted, as long as a sufficient portion of the ITRs remain to allow for replication and packaging functions. These constructs can be designed using techniques well known in the art. (See, e.g., Lebkowski et al. (1988) Molec. Cell. Biol. 8:3988-3996; Vincent et al. (1990) Vaccines 90 (Cold Spring Harbor Laboratory Press); Carter (1992) Current Opinion in Biotechnology 3:533-539; Muzyczka (1992) Current Topics in Microbiol. and Immunol. 158:97-129; Kotin (1994) Human Gene Therapy 5:793-801; Shelling et al. (1994) Gene Therapy 1: 165-169; and Zhou et al. (1994) J. Exp. Med. 179:1867-1875).

Several AAV vectors are available from the American Type Culture Collection (“ATCC”) under Accession Numbers 53222, 53223, 53224, 53225 and 53226.

The AAV vectors can be transfected into a host cell with a helper function, e.g., a helper function plasmid (See Section II) and/or accessory functions to produce recombinant recombinant AAV virions (rAAV). Recombinant AAV virions (rAAV) refer to an infectious, replication-defective virus composed of an AAV protein shell encapsidating a nucleotide sequence encoding a therapeutic protein that is flanked on both sides by AAV ITRs. A number of transfection techniques are generally known in the art. See, e.g., Graham et al. (1973) Virology, 52:456, Sambrook et al. (1989) Molecular Cloning, a laboratory manual, Cold Spring Harbor Laboratories, N.Y., Davis et al. (1986) Basic Methods in Molecular Biology, Elsevier, and Chu et al. (1981) Gene 13:197. Particularly suitable transfection methods include calcium phosphate co-precipitation (Graham et al. (1973) Virol. 52:456-467), direct micro-injection into cultured cells (Capecchi (1980) Cell 22:479-488), electroporation (Shigekawa et al. (1988) BioTechniques 6:742-751), liposome mediated gene transfer (Mannino et al. (1988) BioTechniques 6:682-690), lipid-mediated transduction (Felgner et al. (1987) Proc. Natl. Acad. Sci. USA 84:7413-7417), and nucleic acid delivery using high-velocity microprojectiles (Klein et al. (1987) Nature 327:70-73).

Suitable host cells for producing recombinant AAV virions include, but are not limited to, microorganisms, yeast cells, insect cells, and mammalian cells, that can be, or have been, used as recipients of a exogenous nucleic acid molecule. Thus, a “host cell” as used herein generally refers to a cell which has been transfected with an exogenous nucleic acid molecule. The host cell includes any eukaryotic cell or cell line so long as the cell or cell line is not incompatible with the protein to be expressed, the selection system chosen or the fermentation system employed.

In one embodiment, cells from the stable human cell line, 293 (readily available through, e.g., the ATCC under Accession No. ATCC CRL1573) are preferred in the practice of the present invention. Particularly, the human cell line 293, which is a human embryonic kidney cell line that has been transformed with adenovirus type-5 DNA fragments (Graham et al. (1977) J. Gen. Virol. 36:59), and expresses the adenoviral EIA and E1B genes (Aiello et al. (1979) Virology 94:460). The 293 cell line is readily transfected, and provides a particularly convenient platform in which to produce recombinant AAV virions.

VI Delivery of the Heterologous Protein to the Target Site

The vectors carrying the nucleic acid encoding at least one heterologous protein can be precisely delivered into specific sites of the central nervous system, and in particular the brain, using stereotactic microinjection techniques. For example, the subject being treated can be placed within a stereotactic frame base (MRI-compatible) and then imaged using high resolution MRI to determine the three-dimensional positioning of the particular region to be treated. The MRI images can then be transferred to a computer having the appropriate stereotactic software, and an umber of images are used to determine a target site and trajectory for pharmacological agent microinjection. The software translates the trajectory into three-dimensional coordinates that are precisely registered for the stereotactic frame. In the case of intracranial delivery, the skull will be exposed, burr holes will be drilled above the entry site, and the stereotactic apparatus used to position the needle and ensure implantation at a predetermined depth. The pharmacological agent can be delivered to regions, such as the cells of the spinal cord, brainstem, or brain that are associated with the disease or disorder. For example, target regions can include the medulla, pons, and midbrain, cerebelleum, diencephalons (e.g, thalamus, hypothalamus), telencephalon (e.g., corpus stratium, cerebral cortex, or within the cortex, the occipital, temporal, parietal or frontal lobes), or combinations, thereof.

VII. Delivery of the Therapeutic Agent

The therapeutic agent can be incorporated into pharmaceutical compositions suitable for administration to a subject. Typically, the pharmaceutical composition comprising the therapeutic agent and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody or antibody portion.

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans. The preferred mode of administration is parenteral (e.g., intravenous, subcutaneous, intraperitoneal, intramuscular). In one embodiment, the composition is administered by intravenous infusion or injection. In another embodiment, the composition is administered by intramuscular or subcutaneous injection. In another embodiment, the composition is administered perorally. In another embodiment, the composition is administered systematically.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antigen, antibody or antibody portion) in the required amount in an appropriate solvent with one or a combination of ingredients enumerated above, as required, followed by filtered sterilization.

Generally, dispersions are prepared by incorporating the active compound into a sterile vehicle that contains a basic dispersion medium and the required other ingredients from those enumerated above. In the case of sterile, lyophilized powders for the preparation of sterile injectable solutions, the preferred methods of preparation are vacuum drying and spray-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The vector of the present invention can be administered by a variety of methods known in the art. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdennal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

The pharmaceutical compositions of the invention may include a “therapeutically effective amount” or a “prophylactically effective amount” of the therapeutic agent. A “therapeutically effective amount” refers to an amount effective, at dosages and for periods of time necessary, to achieve the desired therapeutic result. A therapeutically effective amount of the vector may vary according to factors such as the disease state, age, sex, and weight of the individual, and the ability of the therapeutic agent to elicit a desired response in the individual. A therapeutically effective amount is also one in which any toxic or detrimental effects of the vector are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

VIII Uses

(i) Modifying Existing Therapies

For every therapeutic drug, there is a population of individuals that remain non-responsive to the drug. For example, those patients with a low number of receptors, or no receptors, to the therapeutic agent. The invention can be used to alter the environment in a such patients to make the cells more responsive to such existing therapies, thereby improving efficiacy of the therapeutic agent in the population of individuals. The advantage provided by the invention is that there is no requirement for extensive studies time consuming studies into testing the efficiacy and of new therapeutic agents ab initio. Rather, existing therapies shown to be successful in a population of individuals can be used.

(ii) Personalized Medicine

The invention can also be for personalize medicine, whereby a subject that does not have the genetic make-up to be responsive to a therapeutic agent, i.e., does not have the gene that encodes the receptor, is still subject to the method of the invention. This would involve first determining whether the subject has a propensity to express a particular receptor based on their genetic make-up of the subject. After this initial determination, the method of the invention can be used to deliver a protein that modifies the environment of the subject to make the subject responsive to a therapeutic agent. If the subjects; response can be used as a measure to determine how much of the receptor is required to interact with the therapeutic agent, to elicit the desired efficacious response.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety. EXAMPLES

### Example 1

### Materials

(i) Animal Models

A rat model of Parkinson's disease was used as the first example to show the novelty and application of this current invention. This example will utilize the vector rAAV (recombinant adeno-associated vector) to deliver a erythropoietin receptor (EPO-R) expression cassette into non-responsive cells of the SNpc. Expression of EPO-R will promote cell survival of transduced neurons upon systemic application of erythropoietin.

(ii) Growth Factors

Erythropoietin is an ideal growth factor for the protection of dopaminergic neurons. Erythropoietin is well tolerated clinically (Ehrenreich et al. (2002) Mol Med 8, 495-505), can cross the blood-brain barrier when administered systemically (Brines et al. (2000) Proc Natl Acad Sci U S A 97, 10526-10531), and has proven efficacy in protection of both motor neurons (Celik et al. (2002) Proc Natl Acad Sci U S A 99, 2258-2263) and hippocampal neurons (Brines et al. (2000) Proc Natl Acad Sci U S A 97, 10526-10531; Kawakami et al. (2001) J Biol Chem 276, 39469-39475; and Siren et al (2001) Eur Arch Psychiatry Clin Neurosci 251, 179-184; and Siren et al. (2001) Proc Natl Acad Sci U S A 98, 4044-4049) following ischemia. Erythropoietin has demonstrated antiapoptotic, neurotrophic, antioxidant and angiogenic neuroprotective affects in vitro and in vivo (Siren et al. (2001) Proc Natl Acad Sci USA 98, 4044-4049).

(iii) Vectors

The recombinant adeno-associated virus (rAAV) can be utilizing as an example of a vector. Recombinant adeno-associated virus a non-pathogenic parvovirus that has been exploited for gene therapy by the removal of 94% of its genome, allowing insertion of a transgene expression cassette of to approximately 4.5 kilo bases. Long term stable gene transfer and high levels of transgene expression have been demonstrated (Fitzsimons et al. (2002) Methods 28, 227-236) with no associated inflammation and a minimal humoral response (reviewed in Smith-Arica (2001) Curr Cardiol Rep 3, 43-49).

(iv) Immunohistochemistry and Autoradiography

Sometime after vector administration, e.g., four weeks or 2,months, 4, month or 6 months, rats can be overdosed with pentobarbital. Brain immunohistochemistry can be performed as described previously on slide-mounted sections (Young et al. (1999), Nature Med. 5: 448). Following fixation and washing, sections can be incubated overnight at room temperature with a rabbit antibody to a brain antigen present in the neurological disease/disorder. Sections can be washed before application of secondary anti-rabbit antibody. Immunohistochemistry and detection with a secondary antibody can be performed using standard immunofluorescent procedures. Immunofluorescent signals can be captured using a Leica 4d TCS confocal microscope and all images processed using Adobe Photoshop 4.0 (Adobe Systems).

(v) Behavioral Tests

(a) Barnes Circular Maze—This can be carried out as described previously (Barnes et al. (1979) J. Comp. Physiol. Psychol. 93: 74-104). Briefly, rats use spatial navigation to escape from a brightly-lit elevated circular 2 m diameter table which has 18 equally spaced holes around the circumference, one hole which leads to an escape box. On the first day of testing, each rat was placed in the escape box for a four min adaptation period. After one min in the home cage, trial 1 began. On subsequent days, two trials were conducted, separated by one min in the home cage. Testing continued for six days, (11 trials in all). For each trial, rats were placed in the centre of the table under a cylindrical start box for 30 sec, then allowed four min to find and enter the escape tunnel. During this time, the number of incorrect holes searched and latency to enter the tunnel were recorded. All animals spent one min in the tunnel at the conclusion of their trials. Between trials, the table was cleaned with 70% ethanol, and the hole under which the tunnel was placed, though always in the same spatial location, was randomly determined for each rat. From trial 8 onwards, the position of the escape box was altered by 135 degrees, to control for the possibility that rats had learnt to navigate to the escape box by other than spatial means.

(b) Line crossing mobility test—A 2 meter diameter circular table can be divided into 9 segments of approximately equal size. Each rat can be placed in the centre of the table, and allowed 5 min of free movement during which a record was made of the number of times the rat's two front feet crossed a line separating two segments. Testing was conducted for 5 days, (1 trial per day). Between trials the table surface was cleaned with 70% ethanol.

(c) Circular track mobility test—The track used can be a modified version of one used to test mobility in mice. Each rat can be placed inside the track at the start position, facing clockwise, and the number of circuits completed in 5 min was recorded. This procedure can be conducted for 5 days.

(d) Contextual Fear Conditioning—Each rat can be placed in a metal operant chamber (Med Associates Inc.) for 2.5 min of exploration. A tone was then sounded for 30 sec, with a 0.4 mA shock administered during the last 2 sec. 1 h later, rats can be returned to the chamber, and scored for the number of 5 sec intervals spent frozen over a 5 min period. Results can be analyzed with Systat v5.2. (Systat). Two way ANOVA tests can be used, with rat type as the explanatory variable, and day and time (first or second trial of day) as repeated measures where appropriate. Individual trials can be analyzed using a Wilcoxon Rank Sum or two-tailed independent t-test.

### Example 2

### Demonstration of Interventional Pharmacogenomics in a Rodent Parkinson's Disease Model

This example describes using an animal model as an in vivo model to test interventional pharmacogenomics. The most common rodent model for Parkinson's disease is a lesion of the nigrostriatal pathway by unilateral injection of 6-hydroxydopamine (6-OHDA) (Mandel et al., 1999). The extent of the lesion, and the extent of growth factor protection, can be measured by determining the relative levels of contralateral rotation of the rat after administration of amphetamine or apomorphine. Survival of dopaminergic neurons can also be assessed by cell counts of cells that are immunopositive for tyrosine hydroxylase expression in SNpc sections.

Subgroups of rats can be stereotaxically injected with a rAAV vector encoding the EPO-R, or with vehicle alone, into the SNpc. Rats can then be treated with 6-OHDA either before or after addition of EPO and the protective ability of EPO treatment can be determined as described above.

### Example 3

### Screening for Ligands of Mutant EPO-R

Novel ligands for EPO-R can be identified by mutating wild type EPO-R, or a fragment of EPO-R, to produce a mutant EPO-R that retains the ability to dimerize but is incapable of binding wild type EPO. Suitable regions of EPO-R that can be mutated using standard molecular biology procedures as described by Sambrook et al., Supra. The amino acid and nucleic acid sequences of human EPO-R are readily available from Genbank. This mutated EPO-R can be used as bait for selection of a novel peptide that can bind to, and activate it. Ideally, the novel peptide would not be able to bind to or activate wild type EPO-R, as determined by negative selection procedures. This method can be used to identify novel receptor/ligand, that would not be affected by the presence of endogenous EPO ligand, and that would not activate endogenous EPO receptors. The novel receptor/ligand may be used to modify (i.e., activate or deactivate) neuroprotective pathways, thereby altering or ameliorating neurodegenerative diseases and disorders. The method of the invention can be used to alter pathways involving for example, cell proliferation, differentation and growth.

The dimerization of the mutant EPO-R can be initiated by a commercially available dimerizing agent such as those produced by ARIAD. (See e.g., WO 96/06097, WO 97/31898 incorporated herein by reference). Other examples of receptors that can be mutated and used to identify novel ligands are Drosophila extracellular receptors fused to EpoR intracellular which can be used to isolate Drosophila ligands.

1. A method of inducing an efficacious phenotypic response to a therapeutic agent, comprising:
introducing a vector comprising a gene into a mammalian cell, the gene being operably linked to a promoter functional in the mammalian cell and encodes a heterologous protein, wherein expression of the heterologous protein within the mammalian cell modifies the environment of the cell from an environment that is non-receptive to the therapeutic agent to an environment that is receptive to subsequent delivery of the therapeutic agent; and
delivering the therapeutic agent to the mammalian cell, such that the therapeutic agent interacts with expressed heterologous protein and induces an efficacious phenotypic response to the therapeutic agent.

2. The method of claim 1, wherein the heterologous protein is selected from the group consisting of a receptor, an enzyme, and a carbohydrate.

3. The method of claim 1, wherein the therapeutic agent is selected from the group consisting of a ligand, an agonist, antagonist, and drug.

4. The method of claim 1, wherein the vector is selected from the group consisting of adeno-associated viral vector, lentiviral vector, and adenoviral vector.

5. A method of inducing an efficacious phenotypic response to a therapeutic agent in a central nervous system of a subject, comprising:
introducing a vector comprising a gene into a cell present in the central nervous system of the subject, the gene being operably linked to a promoter functional in the central nervous system and encodes a heterologous protein, wherein expression of the heterologous protein within the cell of the central nervous system modifies the environment of the central nervous system from an environment that is non-receptive to a therapeutic agent to an environment that is receptive to subsequent delivery of the therapeutic agent; and
delivering the therapeutic agent to the central nervous system, such that the therapeutic agent interacts with expressed heterologous protein and induces an efficacious phenotypic response to the therapeutic agent.

6. The method of claim 5, wherein the heterologous protein is selected from the group consisting of a receptor, an enzyme, and a carbohydrate.

7. The method of claim 5, wherein the therapeutic agent is selected from the group consisting of a ligand, an agonist, antagonist, and drug.

8. The method of claim 5, wherein the vector is an adeno-associated viral vector selected from the serotype of AAV-1, AAV-2 AAV-3, AAV-4, AAAV-5, AAV-6, and AAV-7.

9. The method of claim 8, wherein the adeno-associated viral vector is AAV-2, or a modified form of AAV-2 with an altered tropism.

10. A method of inducing an efficacious phenotypic response to a therapeutic ligand in a brain of a subject with a disorder, comprising:
introducing a vector comprising a gene into a region of the brain of a subject, the gene being operably linked to a promoter functional in the brain and encoding a heterologous receptor for the ligand, wherein expression of the receptor modifies the environment in the region of the brain from an environment that is non-receptive to the therapeutic ligand to an environment that is receptive to subsequent delivery of the therapeutic ligand; and
delivering the therapeutic ligand to the region of the brain, such that the therapeutic ligand interacts with expressed heterologous receptor and induces an efficacious phenotypic response to the therapeutic ligand.

11. The method of claim 10, wherein the heterologous receptor is a receptor.

12. The method of claim 11, wherein the receptor is a erythropoietin receptor.

13. The method of claim 10, wherein the ligand is erythropoietin.

14. The method of claim 10, wherein the disorder is a neurodegenerative or neurological disorder associated with the brain.

15. The method of claim 14, wherein the neurodegenerative disorder is Parkinson's disease.

16. A method of personalizing medical intervention for a subject with a disorder, comprising:
determining the expression level of a receptor for a therapeutic ligand from a mammalian cell that requires medical intervention;
comparing the expression level of the receptor with a predetermined standard at which a therapeutic ligand is found to be efficacious;
introducing a vector comprising a gene to the mammalian cell that requires medical intervention, the gene being operably linked to a promoter functional in the mammalian cell and encoding a heterologous receptor for the ligand, wherein the gene expresses the receptor for the ligand within the mammalian cell to a level of the predetermined standard, and renders modifies the environment in the mammalian cell from an environment that is non-receptive to the therapeutic ligand to an environment that is receptive to the therapeutic ligand;
delivering the therapeutic ligand to the mammalian cell, such that the therapeutic ligand interacts with expressed heterologous receptor; and
measuring the therapeutic effect of the ligand on the mammalian cell, and modifying the expression level of the receptor to provide personalized medical intervention for the subject.

17. The method of claim 16, wherein the therapeutic ligand is a known ligand.
